(19) United States
(12) Patent Application Publication
(10) Pub. No.: US 2007/0087053 A1
(43) Pub. Date: Apr. 19, 2007

(54) Lozenge for treatment of dry mouth and related conditions

(76) Inventor: Marshall A. Hayward, Bridgewater, NJ (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 11/473,046

(22) Filed: Jun. 23, 2006

Related U.S. Application Data
(60) Provisional application No. 60/726,191, filed on Oct. 14, 2005.

(51) Int. Cl. A61K 36/906; A61K 36/48; A61K 9/46; A61K 36/23

(52) U.S. Cl. 424/466; 424/756; 424/757; 424/725

(57) ABSTRACT

A composition and method for the treatment of dry mouth (xerostomia), comprising a sialogogic compound in combination with an effervescent organic acid-based buffering system.

### FIELD OF THE INVENTION

Two-part tablet or lozenge with a rapid acting effervescent component and long lasting non-effervescent component, and having specific applications to xerostomia and other oral health therapeutics.

### BACKGROUND OF THE INVENTION

Xerostomia (dry mouth) is suffered by an estimated 20%+ of adults (mainly women) as a consequence of the inability to secrete saliva. The condition itself is uncomfortable, but may also have serious consequences, resulting in severe dental decay and oral infections. Dry mouth can be caused by a number of maladies, such as Sjogren's syndrome, diabetes, AIDS, bone marrow transplant or dehydration. It may also occur as a response to radiation treatment or as an unwanted side effect of drug treatments. It is thought that over 1,800 drugs have the capacity to cause dry mouth, when taken over a period of time. Dry mouth may also occur as a physiologic response (e.g. stress, nervousness or “stage fright”) or from other unidentified causes.

Individuals suffering from Sjogren's syndrome or other pathologic causes of xerostomia lack the ability to salivate. However, individuals suffering from the side effects of drug treatments or from diminished salivary gland activity, may benefit from drug treatments such as pilocarpine, which stimulates salivation as well as other secretions such as sweating. In these cases, the curative treatment may be as objectionable as xerostomia itself due to responses such as profuse sweating.

Palliative treatments are generally short acting and cosmetic in nature, and include constant ingestion of water, the use of non-cariogenic candies or demulcents, or other agents to directly hydrate the oral cavity or to provide a lubricious mouthfeel. The individual subject response to such treatments is mixed.

There is anecdotal evidence that some traditional herbal or natural products may stimulate salivation. For the most part, these reports concern the chewing of plant materials—stems, bark, seeds and leaves, etc. Some plants used have broadly stimulating properties, like betel, khat, tobacco and cola nuts. Most have strong tastes: sour as in lemon and citrus peel, bitter like wormwood, golden seal and yarrow stems, or contain unpleasant-tasting irritants like the resins of gum myrrh and the pepper of kava and prickly ash bark.

It is known physiologically that salivation is a reflex response to eating. Saliva obviously lubricates the mouth and also contains bacteriostatic and digestive principles. Strong tasting and dry foods are the most effective in stimulating this response. Salivary stimulation often follows the consumption of distasteful materials, in effect as a defense measure on behalf of the mucosa of the mouth.

Furthermore, maintenance of a moist oral cavity is important for the preservation of good oral hygiene. A dry mouth is susceptible to infection and other conditions (gingivitis, yeast, caries, mucositis, halitosis, and related conditions such as sore throat, and itchy/scratchy throat as in allergic responses), which inadequate mouth pH control and moisture content can exacerbate. Thus, in addition to the direct treatment of xerostomia, these formulations and methods of treatment for dry mouth can complement therapeutic approaches to maintaining good oral health. Therapeutic products for treatment of oral health conditions may benefit form the bilayer sialogogic and demulcent formulation as a platform for delivery of the therapeutic agent.

U.S. Pat. No. 5,804,165 discloses an anti-plaque oral composition using a source of carbon dioxide, silica and xylitol where the carbon dioxide comes from a bicarbonate. The effervescent tablet converts to a solid silica-containing suspension in the saliva of an oral cavity. Related U.S. Pat. No. 5,817,294 discloses a bicarbonate and acid, with silica or other solid materials, in a ratio of 0.32 to 1.0 to 0.8 to 1.0. Related U.S. Pat. No. 5,965,110 discloses the carbon dioxide source and acid with silica and without the use of xylitol, and related U.S. Pat. No. 6,086,854, discloses the carbon dioxide source, the acid, xylitol and a precipitated amorphous silica. These patents all have an insoluble silica material as an abrasive

United States Patent Application 20060057078 describes an effervescent tablet that can be used to carry a variety of functional ingredients to the oral cavity.

Andrews Antacid Tablets® have been manufactured and marketed in Europe for many years, first by Sterling Health, then by SmithKline Beecham (now Glaxo SmithKline) until discontinuation in 2005. These chewable tablets are used to neutralised excess acid and provide relief from acid indigestion, heartburn and trapped wind. They contain calcium carbonate Ph Eur 600 mg, heavy magnesium carbonate Ph Eur 125 mg, malic acid, sugar, magnesium stearate, talc, saccharin sodium, sodium bicarbonate and orange flavor.

### SUMMARY OF THE INVENTION

An object of the invention is to provide a multi-component composition for the treatment of dry mouth (xerostomia), comprising:

a) a first part or layer that rapidly disintegrates in the oral cavity and releases a sialogogic compound, in combination with an effervescent organic acid-based buffering system. and

b) a second part or layer that delivers a demulcent compound into the oral cavity over a period of several minutes and which confers a pH of about 6.4 or above when the composition has been fully consumed.

Another object of the invention is to provide a method for treating dry mouth by administering a multi-component formulation, comprising:

a) a first part or layer that rapidly disintegrates in the oral cavity and releases a sialogogic compound, in combination with an effervescent organic acid-based buffering system. and

b) a second part or layer that delivers a demulcent compound into the oral cavity over a period of several minutes and which confers a pH of about 6.4 or above when the composition has been fully consumed.

Another object is to provide a one-part tablet or lozenge composition to deliver in the oral cavity, comprising a sialogogic compound, and an organic acid and carbonate salt combination that when dissolved together in the oral cavity combine to effervesce, wherein the organic acid is selected from the group consisting of citric acid, fumaric acid, tartaric acid, malic acid, adipic acid and mixtures thereof, and the carbonate salt is selected from the group consisting of sodium carbonate, sodium bicarbonate, potassium carbonate, ammonium carbonate, magnesium carbonate, calcium carbonate and mixtures thereof, the weight ratio of carbonate to acid being in the range of about 1:1 to 1:3, said composition further comprising a flavoring agent, a binder and a lubricant.

### DETAILED DESCRIPTION OF THE INVENTION

A sialogogue or ptyalagogue is an agent that promotes the flow of saliva. In the present invention a sialogogic compound or a sialogogic flavor promotes the flow of saliva.

In the present invention, a demulcent compound or demulcent effect provides a soothing, mucilaginous or oily sensation in the mouth.

A useful sialogogue is the spice cardamom (Elettaria cardamomum). A member of the ginger family, originally from Southeast Asia, it is widely used as a culinary additive in the desert areas of the Middle East, with a traditional reputation as a sialogogue. Seed pods are widely sold in bulk in Arab souks and, among other uses, these are chewed. The primary indication for this traditional remedy otherwise is digestive upsets, including belching and flatulent dyspepsia, conditions that in their own way can have oral implications. Cardamom tastes pleasant enough to be used in some sweet recipes in Middle Eastern and Asian cultures, and is likely to be well tolerated by a modern user. It is relatively unfamiliar to the US and British populations.

Sialogogues may be percieved as flavors or spices, but the physiologic response to a sialogogue does not necessarily depend upon the presence of a flavor per se. Chemicals with no characteristic or distinctive taste may also be effective in stimulating salivation. This is the case with chemical agents such as the OPTAMINT/OPTAFLOW® flavor marketed by Symrise, which can also be used in conjunction with compounds having conventional sialogogic and organoleptic properties.

Stimulation of salivation and retention of moisture in the oral cavity are the objectives of the present methods and formulations The invention is indicated for individuals having the physiologic ability to salivate but do not salivate in sufficient quantity to satisfy their personal comfort level of oral hydration. In contrast to typical confections, we have found that effervescent preparations actively stimulate salivation in many individuals, especially when coupled with selected flavors and aromas. This stimulation of salivation may be directly linked to organic acids such as citrate and tartarate, used in some effervescent preparations. The acids can help buffer the oral cavity and lessen the incidence of fever blisters. Protection against some oral disorders or infections can be improved by maintaining the appropriate pH balance in the mouth, which is typically pH 6-7, preferably about pH 6.4.

This invention specifically discloses a method of treatment for xerostomia and related disorders through a combination of sialogogues, which are active from both a taste (or flavor) response, and a physiologic response to physico-chemical stimulation (as in effervescence). The invention further ameliorates dry mouth by delivering a long lasting demulcent property after the initial stimulation of salivation. The effervescent formulations and the method(s) of manufacturing such formulations are also disclosed.

The present invention is a chewable tablet or hard lozenge for use in the oral cavity. The formulations of the invention specifically combine one or more of cardamom, ginger, licorice, anise, or other sialogogic flavors (such as the plant or herb based products noted above or as flavorless chemicals that are sialogogues) in a naturally sweetened or artificially sweetened effervescent base. The preferred form is a multi-part formulation, however all the ingredients may be contained in a single, relatively homogenous part or layer. The base of the invention is a mildly effervescent combination of acid and a carbonate salt, yielding a composite system that delivers a strong stimulation to the oral cavity and salivary glands to secrete saliva. Preferably the composition is sucked and dissolves slowly, although it can be chewed if desired. Another alternative is to provide granules (or a tablet) that can be dissolved in water and used as a beverage or mouth wash.

The preferred acid component is composed or citric acid, sodium citrate or potassium citrate. Other suitable acids include fumaric acid, tartaric acid, malic acid, adipic acid and other edible acids or their pharmaceutically acceptable salts can be used.

Sodium carbonate and sodium bicarbonate are the preferred carbonate salts. However carbonates and bicarbonates of potassium, ammonium, magnesium, calcium can also be used.

The present sialogogue delivery system generates a buffered solution in the oral cavity which, by maintaining a relatively neutral pH, can help to diminish the formation of caries and renders the oral cavity less prone to infection. The effect is made long lasting through the delivery of agents such as glycerin, polymers such as (but not limited to) natural gums including Xanthan (0.5 to 15% by weight), and 0.2 to 10% by weight of cellulose based materials such as methylcellulose, carboxymethylcellulose, hydroxypropylmethyl cellulose, hydroxyethylcelulose), acrylic acids at 0.2 to 10% by weight, polyethylene glycols at 0.2 to 2 wt %, dextran (0.05 to 0.5 wt. %), gelatin (0.2 to 5 wt. %), polyvinyl alcohol (0.1 to 5 wt. %), polysorbate 80 (0.2 to 2 wt. %), or povidone (0.1 to 4 wt. %).

Sweetener may be chosen from any of the commercially available and pharmaceutically approved sweeteners. Examples of sweeteners are calcium or sodium saccharin, aspartame, acesulfame potassium, sucralose, cyclamates, sucrose, glucose, dextrose, xylitol, manitol and other sugars.

Flavors (including sweeteners) are present in the tablet at 0.1% to 5% by weight, depending on the specific flavor and desired attributes. If natural plant or herb products are incorporated into the formulations to provide sialogogic properties, these may be included at 0.25% to 20% of the composition by weight. Artificial sweeteners are used according to taste, and generally are present at 0.1 to wt. %.

The organic acid components are present in 2 to 15 wt. %/ The effervescent couple (e.g., organic acid, acid salts and bicarbonate) is generally present at between 3 and 20% by weight. The ideal pH of the mouth during dissolution of the effervescent phase is dependent on flavor attributes, but is targeted to pH 3.5 to 5.5. The pH of the mouth after complete dissolution of the total lozenge is about 6.4 to 7.5.

The tablets can be prepared on a standard bilayer tablet press and compressed to a tablet hardness (crushing strength) of approximately 90-150 Newtons, preferably about 100 Newtons. This yields a robust, non-friable tablet. Because effervescence is caused when the acid combines with the carbonate salt to release carbon dioxide, all materials must be anhydrous or nearly so. Pharmaceutical operations (mixing, granulation as necessary, tableting, and packaging) are conducted under conditions of low ambient humidity in order to protect the integrity of the effervescent formulation. In general the water content of the final formulation should be kept below 1 wt. %, preferably below 0.3 wt. %.

The multi-part composition may be physically constructed as a (1) bilayer tablet or lozenge, as (2) a tablet, coated tablet, or a formed lozenge with an outer layer having the rapid acting sialogogic components and an inner core containing demulcent actives, as (3) granules of long lasting demulcent active embedded within a matrix of the sialogogic components (as in a soft confectionery product, a chewing gum, or the like), or (4) any other such configuration that provides both a fast acting component with sialogogic properties and a separate, longer lasting component with demulcent properties.

The total lozenge or tablet mass may range from 400 mg to 2000 mg, depending on particular consumer preferences and desired performance attributes such as lozenge residence time (dissolution) in the mouth. The preferred mass is 800-1250 mg.

In addition to the acid/carbonate salt couple, the composition may contain binders, fillers and flow aids or lubricants. Such tabletting material typically accounts for about 90% of the mass, and comprises mostly sugar alcohols, but includes flow aids, lubricants, flavors and excipients. Suitable binders include sorbitol, lactose, urea, sucrose stearate, starch, maltodextrin, corn syrup solids, sodium citrate, sodium sulfate, sodium chloride, sucrose and dextrates. In general the composition will include either a binder or a lubricant or both.

Suitable excipients include pectin, guar gum, gum arabic, xanthan gum, hydroxymethyl cellulose, hydroxypropyl cellulose, tragacinth gum, alginic acid or salts of alginic acid.

Examples of suitable lubricants are polyethylene glycol, polypropylene glycol, polyvinyl alcohol, polyvinyl pyrrolidone, sodium benzoate, leucine, magnesium stearate, sodium lauryl sulfate, and sodium lauryl sulfoacetate.

In addition to the sialogogic compound, flavor additives may be used such as citrus (lemon, lime, orange grapefruit) berries (raspberry, strawberry, blueberry.) and mint (peppermint, spearmint, wintergreen).

Desensitizing agents may be added such as strontium nitrate and potassium nitrate, to reduce heat or cold sensitivity.

Antimicrobial agents such as cetylpyridinium chloride and domiphen bromide may be added to reduce bacterial levels in the oral cavity.

Breath freshening ingredients such as chlorophyll may be added and prescription medicines such as antibiotics may also be added

### EXAMPLES

The following tablets are prepared under conditions of low ambient humidity using a standard bilayer tablet press, and are compressed to a tablet hardness of approximately 100 Newtons.

Examples and results of dry mouth taste panel for 6 sample executions:

| Component | Sample 1 | Sample 2 | Sample 3 | Sample 4 | Sample 5 | Sample 6 |
|---|---|---|---|---|---|---|
| Polymers or gums, wt % | 1% | 1% | 1% | 1.5% | 2% | 2% |
| (Sialogogic layer) | acrylic | acrylic | acrylic | Xanthan | Xanthan | Xanthan |
| Polymers or gums, wt % | 3% | 3% | 3% | 8% | 10% | 10% |
| (Demulcent layer) | Xanthan | Xanthan | Xanthan | Xanthan | Xanthan | Xanthan |
| Sialogogic Flavors, | Ginger | Mint | Citrus | Ginger | Citrus | Cardamom |
| including sweeteners | 0.6% | 0.3% | 0.3% | 0.8% | 0.9% | 0.7% |
| (total wt %) | | | | | | |
| Non-cariogenic sugar | 85.3% | 81.6% | 87.6% | 79.1% | 73% | 76.2% |
| alcohols (e.g. Xylitol, | | | | | | |
| mannitol, sorbitol - to | | | | | | |
| provide lozenge bulk and | | | | | | |
| aid in compression) total | | | | | | |
| wt % | | | | | | |
| Organic acid e.g. citric | 4.5% | 7.5% | 3% | 5.25% | 7.5% | 5.25% |
| acid (wt %) | | | | | | |
| Sodium Bicarbonate (wt | 1.5% | 2.5% | 1% | 1.75% | 2.5% | 1.75% |
| %) | | | | | | |
| Tablet Excipients (flow | 4.1% | 4.1% | 4.1% | 4.1% | 4.1% | 4.1% |
| aids, lubricants, colors, | | | | | | |
| etc) wt % | | | | | | |
| Nominal Tablet Mass | 850 mg | 850 mg | 850 mg | 850 mg | 850 mg | 850 mg |
| (15 mm diameter) | | | | | | |
| Panel Results* | SS = 70% | SS = 70% | SS = 80% | SS = 70% | SS = 90% | SS = 90% |
| | DE = 50% | DE = 70% | DE = 80% | DE = 90% | DE = 90% | DE = 90% |
| | (C1) | (C2) | (C3) | (C4) | (C5) | (C6) |

*Results of performance evaluation of formulations as judged by a panel of 10 taste/performance evaluators. Responses noted as noticeable Saliva Stimulation (S = Number of 10 as %) and Demulcent Effect (D = Number of 10 as %).
Panel Results - Consensus Comments:
C1: Panel split on flavor suitability (some indicated refreshing, some indicated too bitter). Demulcent effect noticeable but relatively weak.
C2: Refreshing, but flavor intensity weak and less effervescence is preferred. Noticeable demulcent effect.
C3: Refreshing; flavor well received but regarded as weak and more effervescence preferred. Demulcent effect recognized but not pronounced; longer lasting effect desired.
C4: Panel split on flavor suitability (some indicated refreshing, some indicated too bitter). Demulcent effect pronounced.
C5: Strong salivation response and demulcent effects noted. Flavor intensity regarded as very strong, effervescence very strong/aggressive.
C6: Strong salivation response and demulcent effects noted. Generally well received. Participants favored this flavor and tablet.

These tests demonstrate that stimulation of salivation and introduction of a demulcent effect can be achieved within a single product formulation.
Particular embodiments of the present invention have been illustrated and described, however, these are not intended to limit practice of the invention within the scope of the following claims.

1. A multi-component composition for the treatment of dry mouth (xerostomia), comprising:
a) a first part that rapidly disintegrates in the oral cavity and releases a sialogogic compound, in combination with an effervescent organic acid-based buffering system. and
b) a second part that releases a demulcent compound into the oral cavity over a period of several minutes and which confers a pH of about 6.4 or above when said multi-component composition has been fully consumed.

2. The composition of claim 1, wherein said rapidly disintegrating first part is in the form of a layer and said second part is in the form of a second layer.

3. The composition of claim 1, which is a lozenge having an inner core and a rapidly disintegrating outer layer.

4. The composition of claim 1, which is a bi-layer or coated tablet.

5. The composition of claim 1, which has a hardness of at least about 100 Newtons

6. The composition of claim 1, comprising 3-8 wt. % citric acid, 1-3 wt. % sodium bicarbonate, 70-88 wt. % of non-cariogenic sugar alcohol, 1-2 wt. % polymer or gum in the sialogic part and 3-10 wt. % polymer or gum in the demulcent part.

7. The composition of claim 1, wherein the sialogogic compound is selected from the group consisting of cardamom, ginger, licorice, anise and synthetic compounds having
sialogogic properties

8. The composition of claim 7, wherein the sialogogic compound is cardamom (Elettaria cardamomum).

9. The composition of claim 1, which has mass of 800-1250 mg.

10. The composition of claim 7, wherein the sialogogic compound is Symrise Optamint/Optaflow.

11. A method for treating dry mouth by administering a multi-component formulation, comprising:
a) a first part that rapidly disintegrates in the oral cavity and releases a sialogogic compound, in combination with an effervescent organic acid-based buffering system. and
b) a second part that releases a demulcent compound into the oral cavity over a period of several minutes and which confers a pH of about 6.4 or above when said multi-component composition has been fully consumed.

12. The method of claim 11, wherein said rapidly disintegrating first part is in the form of a layer and said second part is in the form of a second layer.

13. The method of claim 11, comprising administration of a lozenge having an inner core and a rapidly disintegrating outer layer.

14. The method of claim 11, comprising administration of a bi-layer or coated tablet.

15. The method of claim 11, wherein the formulation has a hardness of at least about 100 Newtons

16. The method of claim 1, wherein the formulation comprises 3-8 wt. % citric acid, 1-3 wt. % sodium bicarbonate, 70-88 wt. % of non-cariogenic sugar alcohol, 1-2 wt. % polymer or gum in the sialogic part and 3-10 wt. % polymer or gum in the demulcent part.

17. The method of claim 1, wherein the formulation contains a sialogogic compound selected from the group consisting of cardamom, ginger, licorice, anise and synthetic compounds having sialogogic properties including Symrise Optamint/Optaflow.

18. The method of claim 17, wherein the sialogogic compound is cardamom (Elettaria cardamomum).

19. The method of claim 1, wherein the formulation has mass of 800-1250 mg.

20. The method of claim 1, wherein the sialogogic compound is Symrise Optamint/Optaflow.

21. A one-part tablet or lozenge composition, comprising a sialogogic compound, and an organic acid and carbonate salt combination that when dissolved together in the oral cavity combine to effervesce.

22. The one-part tablet or lozenge composition of claim 21, wherein the organic acid is selected from the group consisting of citric acid, fumaric acid, tartaric acid, malic acid, adipic acid and mixtures thereof, and the carbonate salt is selected from the group consisting of sodium carbonate, sodium bicarbonate, potassium carbonate, ammonium carbonate, magnesium carbonate, calcium carbonate and mixtures thereof, the weight ratio of carbonate to acid being in the range of about 1:1 to 1:3, said composition further comprising a flavoring agent, a binder and a lubricant.

23. A tablet or granule composition, comprising: a sialogogic compound, and an organic acid and carbonate salt combination that effervesces when dissolved in water and can by used as a beverage or mouth wash.

24. The composition of claim 1, which is a soft confectionery product or a chewing gum.

25. The method of claim 11, comprising administration of a soft confectionery product or a chewing gum.

26. A soft confectionery product or chewing gum, comprising: an organic acid and carbonate salt combination that when dissolved together in the oral cavity combine to effervesce, and granules of a demulcent compound embedded within a matrix containing a sialogogic compound.
